(19) United States
(12) Patent Application Publication
(10) Pub. No.: US 2005/0177058 A1
(43) Pub. Date: Aug. 11, 2005

(54) System and method for analyzing the brain wave patterns of one or more persons for determining similarities in response to a common set of stimuli, making artistic expressions and diagnosis

(76) Inventor: Nina Sobell, New York, NY (US)

Correspondence Address: EVELYN M. SOMMER, SUITE 825, 250 PARK AVE, NEW YORK, NY, 10022, US

(21) Appl. No.: 11/054,756

(22) Filed: Feb. 10, 2005

Related U.S. Application Data
(60) Provisional application No. 60/543,125, filed on Feb. 11, 2004.

(51) Int. Cl. A61B 5/04

(52) U.S. Cl. 600/545

(57) ABSTRACT

There are provided a system and method for the analysis and presentation of brain wave patterns of one or more persons in response to a common set of stimuli, the persons being present at one or more locations, with the objectives of determining compatibilities, carrying out medical or other diagnostic procedures and producing artistic expressions. The use of a network provides the means for a participation of a large number of subjects in different locations and the broadcasting of the visual and/or oral presentations to a worldwide audience. The system comprises at least one and preferably more EEG headsets, at leas one personal computer, a server running data processing program and at least one audio and/or device video

The priority of provisional application Ser. No. 60/543125 filed Feb. 11, 2004, is claimed.

### BACKGROUND OF THE INVENTION

The present invention relates to a system and a method for the analysis and presentation of brain wave patterns of one or more subjects for the purposes of determining subject compatibility, making artistic expressions and conducting diagnostic procedures. In particular, the invention is related to the analysis of similarities or absence thereof of the brain wave patterns of multiple persons in response toward a common set of stimuli (visual, audio, physical or mental activities and the like), and more specifically, the present invention relates to a system and a method for studying compatibility (habits, hobbies, personalities, etc.) between multiple persons using brain waves collected from such subjects. The present invention also relates to a system and a method for the creation of artistic works (visual, audio, and etc.) using brain waves collected from one or more persons. The present invention further relates to a system and method for use in making a medical diagnosis and as an aid in treatment of mental and medical conditions using EEG produced brain wave patterns. Finally, the present invention also relates to a system and method for truth detection using brain wave patterns. The system and method are preferably deployed on a network allowing the participation of one or more subjects and other participants over a distance. The preferred network is the Internet.

The electroencephalogram (EEG) is a recording of the electrical activity of the brain detected from electrodes pressed against the subject's scalp. The waveforms recorded are generated almost exclusively by inhibitory and excitatory postsynaptic potentials of cortical nerve cells. The nerve cells in the brain produce signals that are called action potentials. These action potentials move from one cell to another across a gap called the synapse. These potentials summate in the cortex and extend through the coverings of the brain to the scalp, where they can be conveniently measured using appropriate electrodes. Rhythmical activity in the routine scalp recorded EEG represents postsynaptic cortical neuronal potentials which are synchronized by the complex interaction of large populations of cortical cells. The fluctuation of surface EEG are produced mainly by the temporal and spatial summation of electrical currents caused by the relatively slow postsynaptic potentials with little or no contribution by the brief action potentials. EEG activity is quite small, measured in microvolts (μV) with the main frequencies of interest up to approximately 30 Hz.

EEG activities can be broken down into four distinct frequency bands: beta activity (>13 Hz), alpha activity (8 to 13 Hz), theta activity (4 to 7 Hz), delta activity (<4 Hz). Beta activity is a normal activity present when the eyes are open or closed. It tends to be seen in the channels recorded from the center or front of the head. Alpha activity is also a normal activity when present in waking adults. It is mainly seen in the channels recorded from the back of the head, and typically has an amplitude of 40 to 100 μV. It is only seen when the eyes are closed and should disappear or reduce in amplitude when the eyes are open. In younger patients, theta activity may be the main activity seen in channels recorded from the back and central areas of the head. Delta activity is only normal in an adult patient if they are in a moderate to deep sleep.

EEG activity is measured using electrodes that are placed on the scalp. These positions are identified by the recordist who measures the head using the International 10/20 System. There is a great variety of electrodes that can be used. The majority are small discs of stainless steel, tin, gold or silver covered with a silver chloride coating. These normally have a lead attached. Alternative methods consist of a cap, a headset, or a headband in which the electrodes are already imbedded.

EEG machines use a differential amplifier to produce each channel or trace of activity. Each amplifier has two inputs. An electrode is connected to each of the inputs. Differential amplifiers measure the voltage difference between the two signals at each of its inputs. The resulting signal is amplified and then displayed as a channel of EEG activity. The manner in which pairs of electrodes are connected to each amplifier of the EEG machine is called a montage. Each montage will use one of three standard recording derivations: common reference, average reference or bipolar.

At its early stages of development, EEG instruments recorded brain waves in an analog manner. Digital EEG systems have subsequently been developed which convert the waveforms into a series of numerical values through a process called analog-to-digital conversion (ADC). The numerical values, containing information about the amplitude, frequency and rhythmicity of various brain waves, can be stored in the computer memory, manipulated and then redisplayed as waveforms on a computer system. If desired, the numerical values can be transformed and presented in a different manner in various formats such as video, audio and so on and the combination thereof. Digitized EEG signals can also be manipulated to change the montage after the recording is made.

The recordation and analysis of EEG have been widely used in the medical field as an aid to diagnose epilepsy, to distinguish between brain death and possible reversible conditions in deeply unconscious patients, to investigate conditions that can affect brain function such as strokes, brain injuries, encephalitis, liver and kidney disease, psychiatric diseases, Alzheimer's disease dementia, etc.

EEG has also been used in biofeedback systems (e.g. U.S. Pat. No. 5,450,855 and references therein), which are employed to monitor and regulate physiological functions and psychological or emotional behaviors, mostly for medical and psychological applications. Brain waves are translated into a visual or auditory display, thereby allowing subconscious behavior of a subject to be monitored and regulated at a conscious level in an interactive manner.

Brain wave monitoring and analysis, particularly biofeedback, have also been used in non-medical applications. U.S. Pat. No. 3,855,998 discloses an entertainment device which measures theta waves in the brain in addition to other physiological data as an indicator of the user's mental state and directs a certain types of audio-visual stimulation to the user to move the user to a desired mental state.

U.S. Pat. No. 5,213,338 discloses a brain wave-controlled amusement device which operates based on the brain wave patterns detected from one or more players.

U.S. Pat. No. 5,253,168 discloses a system for allowing an individual to express themselves in a creative and artistic manner by using biofeedback signals (including EEG) to direct imaging and audio devices. Using a real-time interactive multi-media computerized hardware/software system, a subject is provided with creative and artistic video and/or audio expression feedback relating to the sensed biorhythms. By controlling the relevant biorhythms, the subject can alter the creative expression feedback and use psychological output sensors to reflect thoughts and emotions.

Systems deployed on a network, for example the Internet, for collecting, transmitting and analyzing a person's physiological parameters, such as EEG, are well known for both medical and non-medical applications.

U.S. Pat. No. 6,832,300 discloses a medical device for dispersing maintenance pharmaceutical drugs. EEG, heart rate and other physiological parameters representing a patient's conditions can be collected using appropriate sensors and then transmitted to a remote site via a network, such as the Internet, for review by a heath care provider. Based on the interpretation of the patient's conditions, appropriate medical instructions are transmitted back to the patient's site and trigger an appropriate drug delivery system.

U.S. Pat. No. 6,510,340 discloses a method and apparatus for electroencephalography wherein EEG readings from a patient are transmitted via a network (such as the Internet) to one or more remote readers for extensive control by the experts located offsite.

U.S. Pat. No. 5,791,342 discloses a medical data transmission system wherein various physiological parameters (such as EEG) of a patient are collected, first digitized and processed locally, then transmitted via a network (such as the Internet) to a remote location for further processing and presentation.

U.S. Pat. No. 6,792,304 discloses a method and a system for mass communication assessment. A cognitive task is transmitted from a central control site to a plurality of remote test sites via Internet. The brain response (such as EEG readings) of the subjects at the remote sites in response to the task is recorded and transmitted back to the central control site via the Internet. The central control site then computes the variations in the brain activities for the subjects at each of the selected sites.

The use of EEG measurement for lie detection is also known. U.S. Pat. No. 5,406,956 discloses a method and an apparatus for truth detection by recording and analyzing electrical brain responses. Other physiological parameters that reflect autonomic nervous system (ANS) activity (e.g. blood pressure, heart rate, sweating) have been traditionally used for lie detection.

### SUMMARY OF THE INVENTION

It is an object of the present invention to provide a system and a method by means of which EEG readings from more than one subject at the same or different locations are collected at the same time or otherwise, analyzed and compared, when they are exposed to a common set of stimuli.

It is a further object of the present invention to provide a system and a method by means of which the compatibility (habit, hobby, personality and etc.) of more than one subject can be studied using their EEG readings when a common set of stimuli are presented to them.

It is yet a further object of the present invention to provide a system and a method where such EEG readings can be carried out in a delocalized environment and the data collection, transmission, and analysis are carried out in a networked environment.

It is yet another object of the present invention to provide a system and a method by means of which one or more subjects can use their EEG readings to express themselves in an artistic manner and produce artistic work.

It is yet another object of the present invention to provide a system and a method by using EEG readings by means of which concealed information can be discovered or verified from one or more subjects.

It is to be noted that the physiological data collected and analyzed for the above-mentioned purposes are not limited to EEG readings. It is within the intent of the invention to use as indicated other physiological data such as heartbeat, galvanic skin response, EKG, and the like and the combination thereof.

These and other features of the invention will become clearer with reference to the following detailed description of the presently preferred embodiment and drawing.

### BRIEF DESCRIPTION OF THE DRAWING

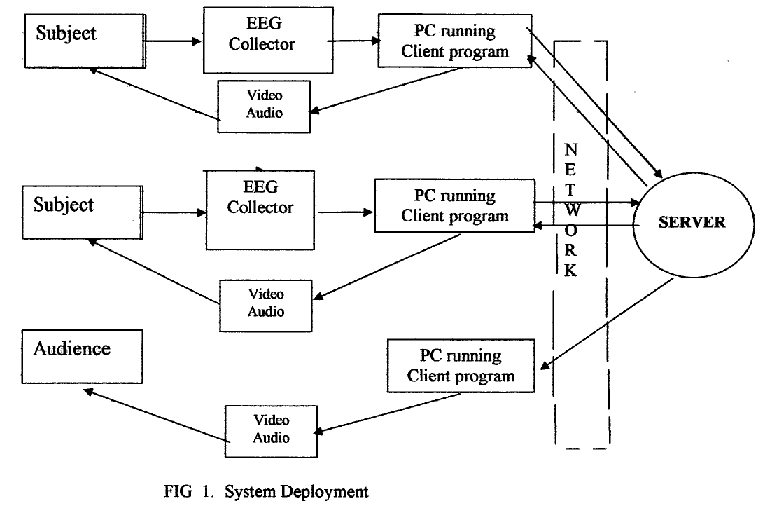

In FIG. 1, there is illustrated in block diagram form a server/client based system for receiving EEG signals from more than one subject and providing video and/or audio feedback to the subjects involved and to the audience.

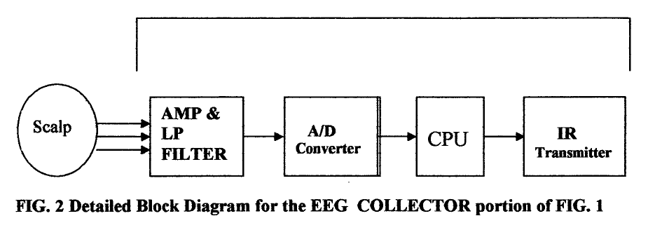

In FIG. 2, there is illustrated in block diagram form the EEG COLLECTOR portion of FIG. 1 for collecting EEG signals.

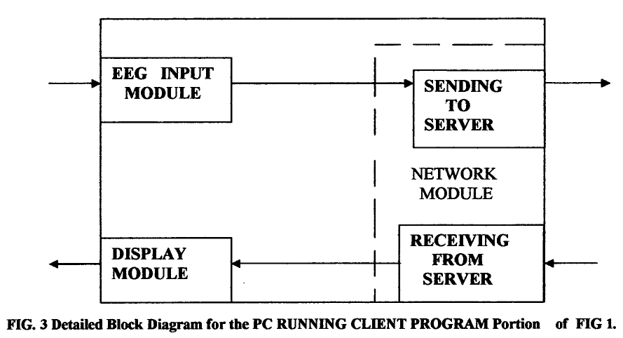

In FIG. 3, there is illustrated in block diagram form the PC RUNNING CLIENT PROGRAM portion of the system of FIG. 1 for processing EEG signals, processing inputs from the server and generating electrical signals for driving the video and/or audio feedback mechanisms.

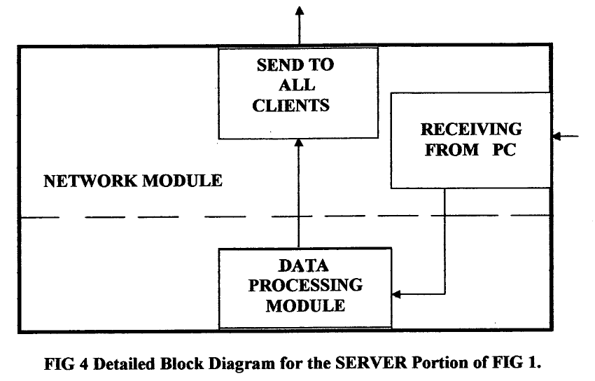

In FIG. 4, there is illustrated in block diagram form the SERVER portion of the system of FIG. 1 for data processing and broadcasting of the processed data to all PCs.

### DETAILED DESCRIPTION OF THE INVENTION

In accordance with the invention, a system is provided wherein one or more than one subject at the same or different locations can have their EEG signals recorded at the same time or otherwise, analyzed, compared and presented back to them and/or an audience or the coordinator of the procedure in a video and/or audio format. The changes in their EEG signals as a function of a common set of changing external stimuli (such as the exposure to a musical track, a painting, etc.) or when the subjects are performing a common set of physical or mental activities can be followed and compared to study the compatibility of their habits, hobbies, personalities, etc. The temperament results can be commercially used to facilitate psycho-sociological interaction, matchmaking, friend-searching or for other psychological, medical, recreational, security or educational purposes.

The video and/or audio presentation of the EEG reading from one person or the combined EEG readings from multiple subjects, can also be used for artistic purposes, such as generating artistic works or providing an artistic experience to the subjects involved and/or an audience. The use of network provides the means for the participation of a large number of subjects in different locations and the broadcasting of the visual and/or aural presentations to a large audience all over the world.

To these ends, the present invention provides a computerized, server/client PC (Personal Computer) based, networked system for the collection, analysis, and comparison and/or integration of EEG readings.

As can be seen from FIG. 1, the EEG readings from multiple subjects (exemplified by two) are each fed into a personal computer on-site. If any two or more subjects are grouped at the same location, a single computer for these subjects might be used. The EEG data are relayed through a network to a server. The network can be any viable computer network, notably an Intranet within an organization or the Internet on the global scale. The server processes the EEG data for the comparison of EEG patterns if the compatibility is desired, or for the generation of an integrated EEG data if a collaborative artistic work or artistic experience is desired. The processed data are further sent back to the local PC computers close to each subject, where it is ultimately passed on to the video and/or audio modules to provide a feedback to the subjects or a presentation to the audience.

FIG. 2. Illustrates how the system is used for collecting EEG readings from the subjects. The number and positions of the electrodes (on the scalp of the subject, not shown in FIG. 2) for collecting EEG readings can be in any viable manner as known in the art. The electrodes are housed in a headset/headphone/headband for convenient and comfortable wearing. Additional components included in the head unit are amplifier, filter, analog-to-digital converter, a microprocessing unit, and data transmission unit. The earlobe is the shield/ground point. Another (or the same) earlobe clip can be used optionally to measure heartbeat. A reference electrode is placed on the occipital bone behind the ear. The remaining monitor electrode is placed on the scalp overlaying the cerebrum. The headset will have convenient default electrode placement and ‘advanced user’ plug in remote electrodes for monitoring other areas of brain activity and the monitor electrode may alternatively be placed on other regions of the scalp to measure the local EEG. The monitor and reference electrodes may actually be placed anywhere on the scalp. The measurement is the differential between the two electrodes. All relevant EEG waves are monitored: alpha, beta, delta, and theta. The EEG waves are amplified and filtered to remove artifacts. An analog-to-digital converter is used to convert the analog EEG signals collected to digital signals, which can be processed using computers. Controlled by a microprocessor, the digital EEG signals are relayed to the transmission unit for passage to the personal computer. The transmission can be achieved using wires, or wirelessly (infrared, radio frequency, etc.). The preferred method is using an IR emitter for sending the data, and the data is collected by the personal computer equipped with an IR receiver, which is either built into the computer or connected to the personal computer through a suitable connection such as another IR, or RS-232 or the like. Optionally, the headphone can be equipped with an IR receiver and the PC equipped with an IR emitter which transmits data back to the headphone unit. using visual and/or aural presentations. When the visual and/or aural aids are used, both drawing, color schemes and sound schemes can shift with the proportion and characteristics of the four types of the brainwaves. If the collaborative creation of artistic work is the desired task, the software is responsible for creating visual and/or aural expressions based on the proportions and characteristics of each of the four brainwaves. The visual image generated from EEG can be overlaid or integrated further with other images, such as a video clip of the subjects. The audio tracks generated from EEG can be overlaid or integrated further with other sound tracks.

It is to be noted that the use of network allows for the simultaneous participation of a large number of subjects at different locations and the broadcasting of the visual and aural expressions to a large audience all over the world.

The way the EEG data is presented, either visually or aurally, can be significantly different depending on the number of subjects.

When multiple persons are involved in the analysis of similarities in brain wave patterns or the creation of artistic works, participating subjects each wears a headset which collects the EEG readings using electrodes placed on their scalps. The EEG analog readings are converted into digital signals inside the circuits of the headset and transmitted by wire or preferably wirelessly to a PC close to the subject. The PC forwards the digital EEG data via network to a server. The server receives multiple inputs from all the PCs and uses the data processing program developed by the inventor for finding the

FIG. 3 depicts the construction of the PC RUNNING PROGRAM portion of FIG. 1. The EEG data transmitted from the headphone enters the PC through in the EEG INPUT MODULE via IR, RS-232 or other suitable connection, and is thereafter passed through the NETWORK MODULE before it is sent to the SERVER via network. The NETWORK MODULE is also responsible for receiving the processed data from the SERVER via network and for passing the data onto the DISPLAY MODULE. The DISPLAY MODULE further sends the data to the Video/Audio unit for presentation to the subjects or audience.

The PC can be designed to be a simple data relay instrument between the EEG headset and the server, and between the server and video/audio equipment. Alternatively, the PC can be designed to take a more active role, such as performing a preliminary EEG data analysis before the data is transmitted to the server, and graphics/sound manipulation before the data is sent to the video/audio devices. Each PC can also handle more than one subject if they are located at the same place. The video device can be a computer monitor, TV, or other professional image projection devices. The audio device can be the computer speakers or externally attached sound systems.

FIG. 4 depicts the construction of the SERVER portion of FIG. 1. EEG data from each individual PC is received via network by the server, and then passed onto a DATA PROCESSING MODULE, where the EEG readings from multiple subjects are analyzed, compared, or integrated using a proprietary program. The processed data is then sent to the PCs for all of the clients/subjects and the audience.

The EEG signals from each subject are continuously monitored under different conditions, such as when the subjects are serene, or exposed to a common set of stimuli, for example, the same musical tracks, photographs, foods and/or TV programs. Alternatively the subjects can be engaged in a predetermined set of physical or mental activities.

The presence of the SERVER allows for a real-time centralized data processing. The proprietary software for data processing in the SERVER portion is responsible for generating visual and aural displays from the EEG readings, for comparing and searching for similarities of the EEG patterns of multiple subjects, for generating artistic video/audio works from one person's EEG reading or the integrated EEG signals from multiple subjects. Using the software at the server (or, if desirable, the software running on the PC), the EEG signals are broken down to each individual component wave (alpha, beta, delta and theta) with different frequencies and physiological significances. If the compatibility is the desired information from the examination, the intensity, frequency and rhythmicity of each component wave are examined for comparison purpose, and the software is capable of identifying which EEGs most closely resemble each other. A resemblance of EEG patterns when presented with the same stimuli indicate an enhanced likelihood of compatibility of the hobbies, habits, personalities, etc. between the subjects. The level of resemblance can be presented as a numeric value, or in an artistic format similarities in their brain wave patterns, or for generating a composite video and/or audio art based on the brain wave inputs, depending on the nature of the task requested by the subjects or their coordinator. The server further forwards the processed data back to each PC, which is located close to the subject and the audience via network. After receiving the processed data, the PC will relay the data to drive a video and/or audio system to provide a feedback to the subjects.

When only one person is involved in the creation of an artistic work, the subject can still use the networked system or the system can be simplified to exclude the networking units and the server. The PC running client program then is equipped with software for processing the EEG readings and to drive the video and/or audio feedback units.

1. A system for the collection, analysis and comparison of EEG brain waves of more than one subject, comprising
a. more than one EEG headsets,
b. at least one personal computer,
c. a server running data processing program, and
d. at least one video or audio device or both.

2. A system according to claim 1 wherein the said EEG headsets further comprise electrodes, analog-to-digital converter, a microprocessor, and a data transmission unit.

3. A system according to claim 2 wherein the said data transmission unit is an infrared data transmission unit.

4. A system according to claim 1 wherein the said personal computer and the said server communicates via network.

5. A system according to claim 4 wherein the said network is the Internet.

6. A system according to claim 1 wherein said data processing program determines the similarity between brain wave readings from multiple subjects.

7. A system according to claim 1 wherein said data processing program generates composite video/audio data by combining brain wave readings from multiple subjects.

8. A system for the collection, analysis and transmission of EEG brain waves of a single subject for presentation of the data to at least one video and/or audio device comprising
a. an EEG headset,
b. a personal computer
c. a server running a data processing program, and
d. at least one video and/or audio device.

9. A method for comparing the similarity of brain wave readings from multiple subjects comprising the following steps,
a. collection and digitization of EEG brain wave readings from more than one subjects using headsets,
b. transmission of the digital EEG signals to personal computers,
c. centralization of the digital EEG signals on a server from the personal computers,
d. analysis and comparison of the digital EEG signals on the server using computer programs,
e. distribution of the data generated by the server based on EEG analysis to the personal computers, and
f. presentation of the data received from the server in video/audio formats to the subjects and audience.

10. method according to claim 9 wherein the transmission of EEG brain wave signal to the said personal computers is performed using infrared communications.

11. method according to claim 9 wherein the centralization of the digital EEG signals on the said server from the said personal computers is performed via the network.

12. a method according to claim 11 wherein the said network is the Internet.

13. a method according to claim 9 wherein the distribution of data from the said server to the said personal computers is performed via the network.

14. a method according to claim 13 wherein the said network is Internet.

15. a method for generating artistic works based on brain wave readings from one or more subjects comprising the following steps,
a. collection and digitization of EEG brain wave readings from one or more subjects using headsets
b. transmission of the digital EEG signals to personal computers,
c. centralization of the digital EEG signals on a server from the personal computers,
d. integration of the digital EEG signals on the server using computer programs,
e. distribution of the data generated by the server based on EEG analysis to the personal computers,
f. presentation of the data received from the server in video/audio formats to the subjects and audience.

16. a method for generating artistic works based on brain wave readings from one subject comprising the following steps,
a. collection and digitization of EEG brain wave readings from one subject using headset
b. transmission of the digital EEG signal to a personal computer
c. processing of the digital EEG signal on the said personal computer
d. presentation of the data generated on the personal computer in video/audio formats to the subject.

17. A system according to claim 5 wherein said system is used to evaluate the compatibility of two or more persons.

18. A system according to claim 5 wherein said system is used to create an artistic work.

19. A system according to claim 1 wherein said system is used to making a medical diagnosis.

20. A system according to claim 1 wherein said system is used in a truth detection procedure.
